(19) United States
(12) Patent Application Publication
(10) Pub. No.: US 2014/0330285 A1
(43) Pub. Date: Nov. 6, 2014

(54) TISSUE RETREIVAL BAG FOR REMOVAL OF TISSUE

(71) Applicant: Rosenblatt Associates, LLC, West Newton, MA (US)

(72) Inventors: Peter L. Rosenblatt, West Newton, MA (US); Anthony J. DiSciullo, Westwood, MA (US)

(73) Assignee: ROSENBLATT ASSOCIATES, LLC, West Newton, MA (US)

(21) Appl. No.: 14/359,214

(22) PCT Filed: Nov. 19, 2012
(86) PCT No.: PCT/US2012/065859

§ 371 Date: May 19, 2014

Related U.S. Application Data
(60) Provisional application No. 61/561,403, filed on Nov. 18, 2011.

(51) Int. Cl. A61B 17/00

(52) U.S. Cl. CPC A61B 17/00234; A61B 2017/00287
USPC 606/114

(57) ABSTRACT

Devices and methods for removal of tissue with a tissue retrieval bag are disclosed.

CROSS-REFERENCE TO RELATED APPLICATION

This application claims the benefit of U.S. provisional application Ser. No. 61/561,403 filed 18 Nov. 2011, which is hereby incorporated herein by reference in its entirety.

### FIELD

The disclosed systems and methods relate generally to systems and methods for performing removal of tissue with a retrieval bag.

### BACKGROUND

Surgery to remove masses in the abdomen, such as a uterus or myoma, may be performed in a minimally-invasive manner, which usually involves morcellating the tissue and removing it through an abdominal port site incision. For example, laparoscopic supracervical hysterectomy involves mobilization of the bladder off the uterus, control of vascular pedicles (specifically the uterine and ovarian arteries), transection of the uterus from the cervix, and then removal of the uterus from the abdominal cavity. This last step is most often performed with the use of an electromechanical morcellator. Strips of uterine tissue are drawn through the morcellation device until the entire specimen has been removed. One of the consequences of electromechanical morcellation is the spreading of small pieces, or “chips” throughout the abdominal cavity, which the surgeon tries to collect before closing the abdominal incisions. There is ample literature describing untoward outcomes that have resulted from surgeons failing to remove all these chips, including parasitic myomas and worsening endometriosis. Unexpected uterine malignancies that have been morcellated have also been reported, resulting in iatrogenic spread of tumors throughout the abdomen.

### SUMMARY

The present disclosure provides systems and methods for performing morcellation of masses within a retrieval bag. In one embodiment, the procedure involves insertion of an endoscopic bag into the abdominal cavity. The bag has an opening on one side, into which the specimen to be removed (such as the uterine fundus, myoma(s) or ovary) is placed with the assistance of other endoscopic instruments. At this point, the bag is closed; this may involve cinching down a purse-string type closure or other closure method that maintains a tight seal to prevent the loss of gas and tissue contents. The endoscopic bag may be translucent or opaque. The endoscopic bag would have one or more ports built into the surface of the bag. These ports may be flush with the bag surface, or may be slightly elevated off the surface. The ports may have tabs coming off the port, which may assist in steadying the bag while inserting instruments into the bag. The ports may have valves of some sort that allow introduction of the instruments but prevent loss of pressure inside the bag when instruments are removed. The bag may have an accessory port for insertion of gas to distend the bag or gas may enter the bag through the same port as one of the laparoscopic instruments. The bag may have a suture attached (such as part of the purse string) that could be used to suspend the bag through one of the laparoscopic ports.

An electromechanical morcellator and other instruments are inserted through ports on the endoscopic bag. The laparoscope may be placed into the bag through one of the other ports. If the cannula of the laparoscope is also placed within the bag, and if the insufflation is being delivered through this same port, the insufflation will distend the endoscopic bag. Alternatively, insufflation can be performed through a different port, and possibly one specially designed to accept an attachment for gas tubing. A tenaculum or other grasping instrument is placed through the morcellator and grasps the tissue within the bag. Another instrument may be placed through an additional port on the endoscopic bag and may be used to “feed” the tissue to the surgeon controlling the grasper or tenaculum coming through the morcellator. In this method, the morcellation is performed within the distended endoscopic bag, and this is performed under direct visualization with the laparoscope. Alternatively, there may not be a need for a second grasping instrument (besides the tenaculum) as the inner contour of the endoscopic bag may steady the specimen for the operator using the morcellator and tenaculum. Following removal of most or all of the tissue, the instruments are removed from the endoscopic bag and the bag may be removed from the abdominal cavity. Since the bag should be essentially empty of specimen after morcellation, there is no need to enlarge the port site as long as the excess gas has been removed from the bag.

### BRIEF DESCRIPTION OF THE DRAWINGS

These and other features and advantages of the systems and methods disclosed herein will be more fully understood by reference to the following detailed description in conjunction with the attached drawings in which like reference numerals refer to like elements through the different views. The drawings illustrate principles of the systems and methods disclosed herein and are not necessarily to scale. Implied absolute or relative dimensions are not limiting but are instead provided for illustrative purposes.

FIG. 1 demonstrates one design of the instrument, which consists of an endoscopic bag with an opening for insertion of tissue, and a method of closure of the opening in the bag. Several ports for placed on the outer surface of the endoscopic bag are shown.

FIG. 2 illustrates the instrument described above with an alternative method of bag closure and several ports placed in the outer surface of the endoscopic bag

FIG. 3 demonstrates another embodiment of the instrument described above with several ports for endoscopic instruments and a separate port into which the laparoscope is placed and is recessed into the bag, but does not actually enter the bag. This would prevent debris from smudging the end of the laparoscope.

FIG. 4 illustrates the endoscopic bag described above with a laparoscope, morcellator and grasper inserted into the ports.

FIG. 5 illustrates the bag described in FIG. 4 inside an abdominal cavity.

### DETAILED DESCRIPTION

For clarity and convenience, a number of exemplary embodiments will be described relating to a particular anatomic site, the female pelvis. However, it will be readily apparent to one of ordinary skill in the art that the disclosed systems and methods may be employed in a wide variety of anatomical settings to treat a broad range of abnormalities.

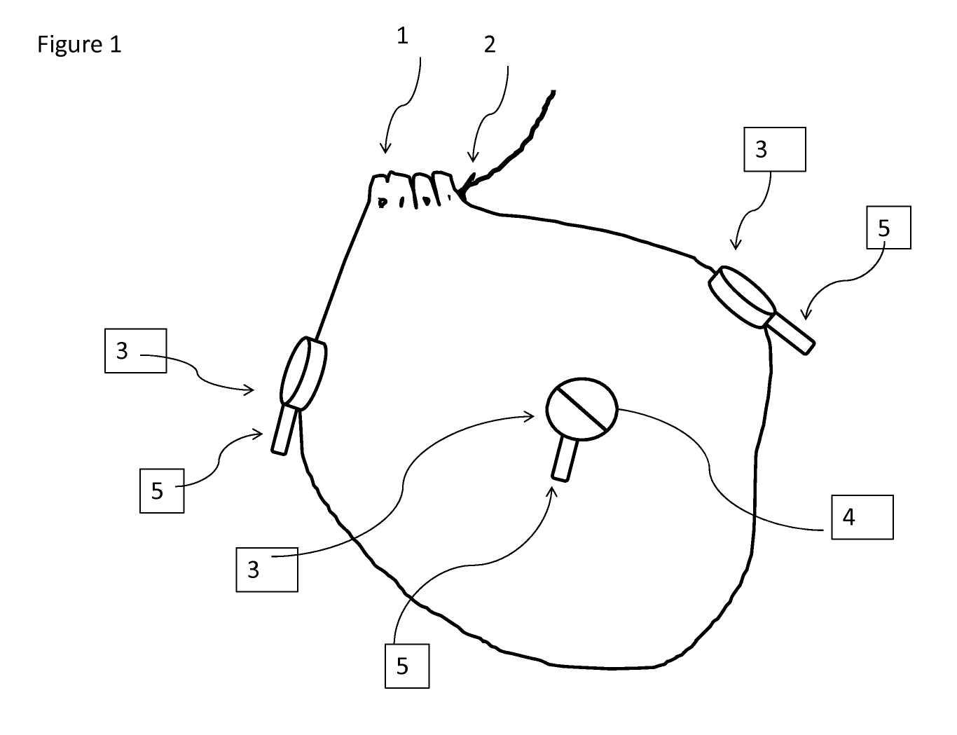

FIG. 1 illustrates the general design of the endoscopic bag with an opening on one side of the bag 1 into which the specimen to be removed is placed. Following placement of the specimen, this opening may be cinched closed by pulling on the purse string suture that has a pre-formed knot 2. The outer surface of the bag has several instrument ports 3 that have openings for endoscopic instruments to be placed within the bag. The ports may have valves 4 that prevent loss of gas from the bag. The ports may have tabs 5 that may be grasped by endoscopic instruments to assist getting other instruments into the bag through the ports.

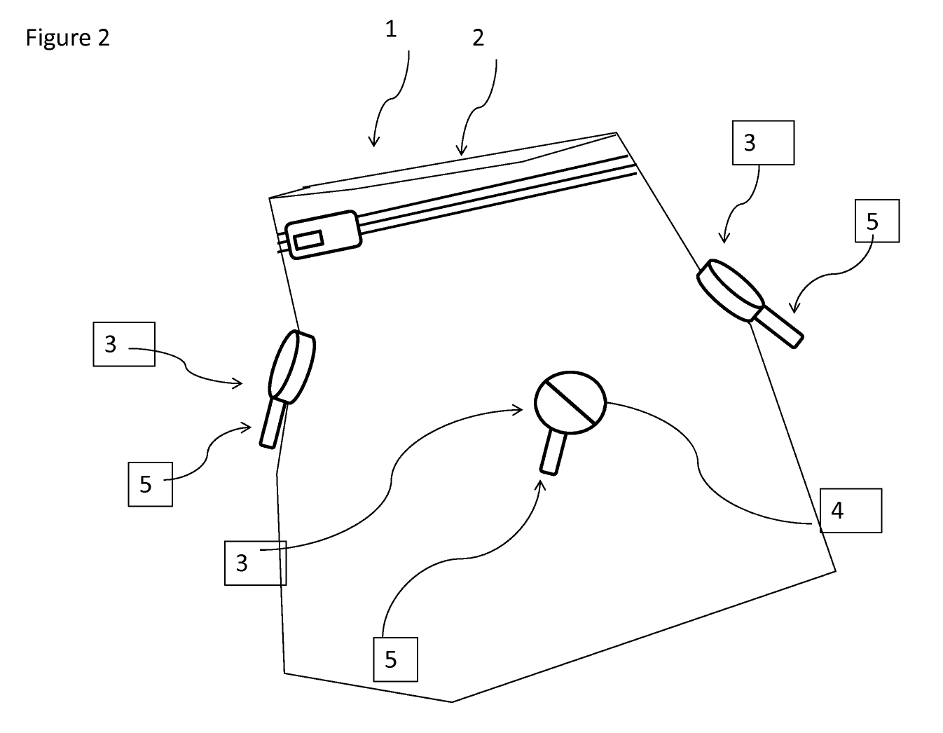

FIG. 2 illustrates the instrument described above with an alternative method of bag closure, which is a “zip-lock” configuration 6. The bag is inserted with the opening in the open position and once the specimen is placed within the bag, the surgeon moves the “zipper” to the closed position. The bag is then inflated through one of the ports on the side of the bag.

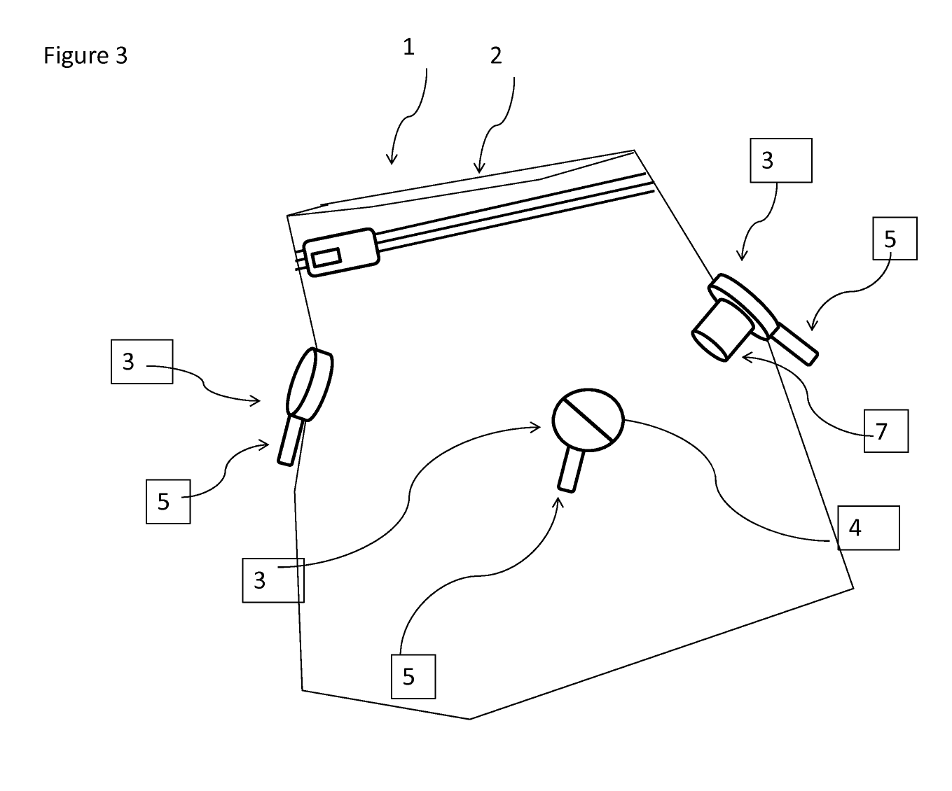

FIG. 3 illustrates another configuration of the endoscopic bag, whereby one of the ports on the outer surface of the bag is specifically designed for the laparoscope to be placed into a recess 7 within the bag, but the laparoscope does not actually enter the bag and therefore does not get smudged from endoscopic contents.

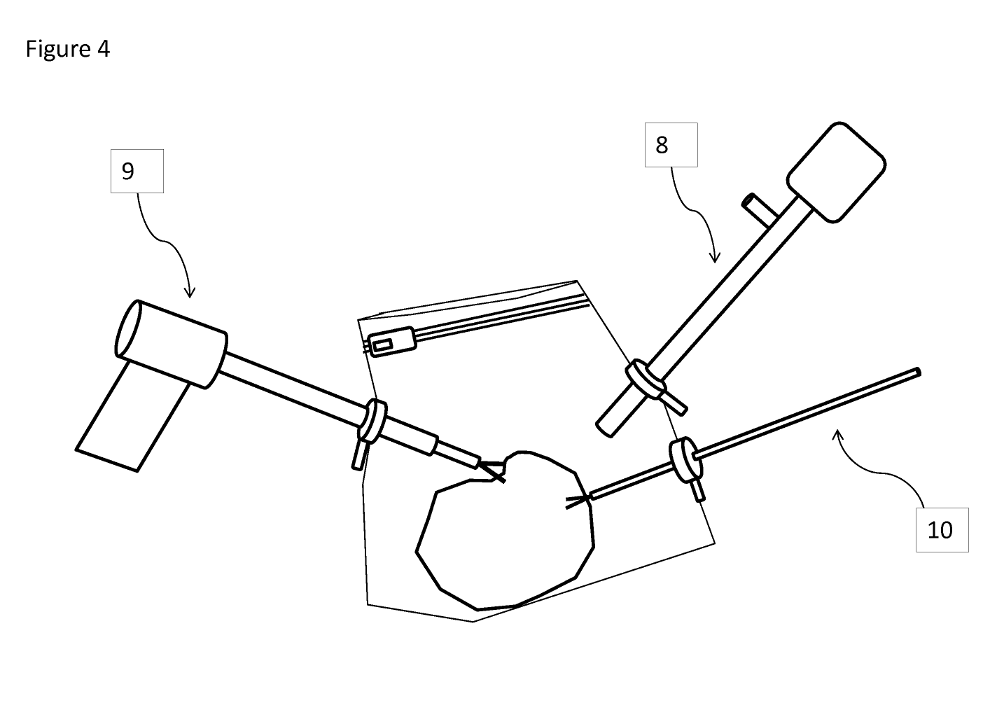

FIG. 4 demonstrates the endoscopic bag with the laparoscope 8, the morcellator 9 with a tenaculum placed within 10 and another grasping instrument 11.

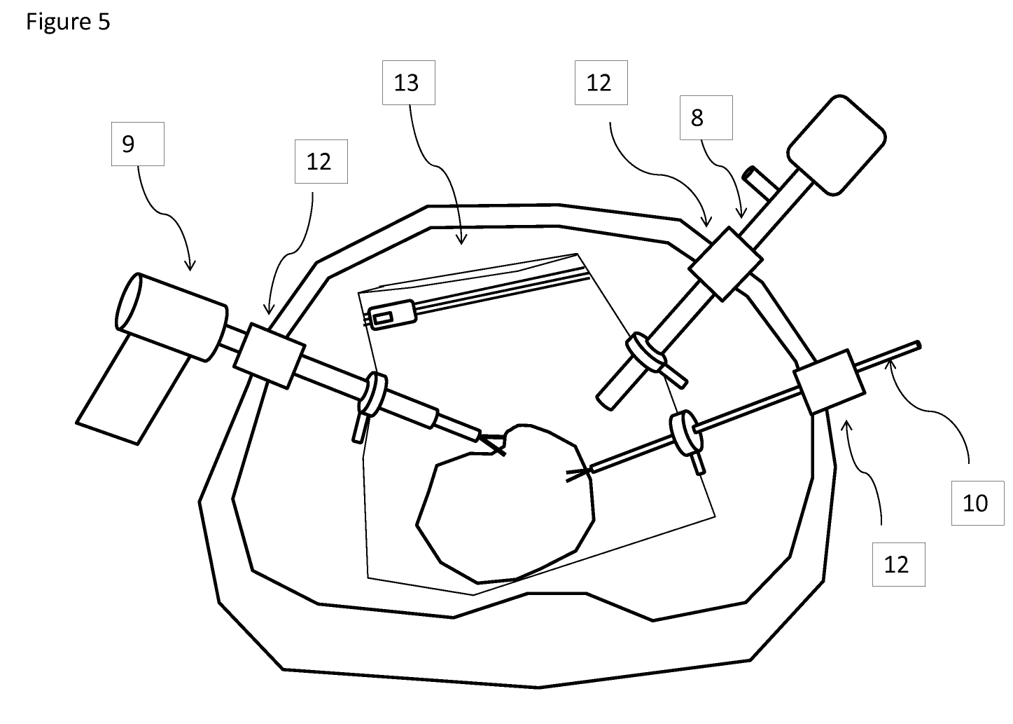

FIG. 5 illustrates the bag described in FIG. 4 inside an abdominal cavity 13 with trocar cannulas 12 that permit insertion of endoscopic instruments.

More generally, a tissue retrieval bag may define an opening to receive tissue. The bag may include a closure adjacent to the opening. The closure may be capable of transitioning between a first state in which the opening admits the passage of tissue, and a second state in which the opening is substantially sealed against the passage of tissue. The bag may also include a first port configure to allow the passage of the working end of a surgical instrument from outside the bag to inside the bag. The bag could include one or more additional ports. The ports could be identical, or could be configured to receive different instruments. The closure could be, for example, a zipper or a drawstring.

In addition to sealing against the passage of tissue, the closure can also seal the opening against the passage of gas. The bag may include an insufflation port that is configured to make an airtight seal with a source of pressurized air. When the opening is substantially sealed against the passage of gas and the insufflation port is connected to a source of pressurized air, the bag can be inflated.

The bag may also include a port or ports that have a tab. The tab or tabs may allow a laparoscopic surgeon to more easily grasp and maneuver the bag. The port or ports may also include a valve configured to seal around a surgical instrument so that, when the working end of the instrument is inside the bag, the valve's seal makes the port substantially airtight.

A bag with any or all of the above features in any combination can be used as follows. A surgeon may access a body cavity where a tissue is to be removed. Accessing the body cavity may, for example, involve making an incision to access the abdominal cavity, or could be done endoscopically, for example by accessing the stomach or bowel. The bag can be placed inside the body cavity. The tissue to be removed can be freed from its surroundings and moved into the bag through the opening of the bag. The tissue can be moved into the bag in entire pieces without prior morcellation. For example, if the operation is a hysterectomy and the tissue to be removed is the patient's uterus, the entire uterus may be placed inside the bag whole. Where the operation is an oophorectomy and the tissue to removed is the patients ovaries, both whole ovaries may be placed in the bag prior to morcellation. The bag can then be closed, sealing the tissue inside. A morcellator can then be inserted into the bag through the first port. The tissue to be removed can then be morcellated inside the bag. The morcellated tissue can then be removed from the bag through a cannula to the exterior of the patient. In this way, the morcellated tissue is always contained, either within the bag or the cannula, until it is outside the patient. Finally the bag can be removed from the patient.

We claim:

1. A tissue retrieval bag defining an opening to receive tissue, the bag comprising:
a closure adjacent to the opening, the closure capable of being transitioned between a first state in which the opening admits the passage of tissue, and a second state in which the opening is substantially sealed against the passage of tissue; and
a first port configured to allow the passage of a working end of a surgical instrument from outside the bag to inside the bag.

2. The bag of claim 1 comprising at least one additional port configured to allow the passage of the working end of a surgical instrument from outside the bag to inside the bag.

3. The bag of claim 1 wherein the closure is a zipper.

4. The bag of claim 1 wherein the closure is a drawstring.

5. The bag of claim 1 wherein:
when the closure is in the first state, the opening admits the passage of gas; and
when the closure is in the second state, the opening is substantially sealed against the passage of gas.

6. The bag of claim 5 further comprising an insufflation port configured to make an airtight seal with a source of pressurized air so that when (a) the closure is in the second state, and (b) the source provides pressurized air to the insufflation port, then the bag is inflated.

7. The bag of claim 1 wherein the first port comprises a tab.

8. The bag of claim 1 wherein the first port comprises a valve configured to seal around a surgical instrument so that, when the working end of the instrument is inside the bag, the first port is substantially airtight.

9. A method of retrieving tissue from a patient comprising:
accessing a body cavity of the patient, the cavity containing a tissue to be removed from the patient;
placing the bag of any of claims 1-8 in the cavity; wherein the closure of the bag is in the first state and the opening admits the passage of tissue;
freeing the tissue to be removed and moving the freed tissue into the bag through the opening;
transitioning the closure into the second state so that the opening is substantially sealed against the passage of tissue;
inserting a morcellator into the bag through the first port;
morcellating the tissue to be removed with the morcellator, while the tissue to be removed remains inside the bag;
removing the morcellated tissue from the bag through a cannula to the exterior of the patient;
removing the bag from the patient.
